(19) United States
(12) Patent Application Publication
(10) Pub. No.: US 2012/0063976 A1
(43) Pub. Date: Mar. 15, 2012

(54) METHODS OF REMOVING CARBON DIOXIDE AND SORBENTS FOR SAME

(75) Inventor: Michael T. Benson, Idaho Falls, ID (US)

(73) Assignee: BATTELLE ENERGY ALLIANCE, LLC, Idaho Falls, ID (US)

(21) Appl. No.: 12/878,853

(22) Filed: Sep. 9, 2010

(51) Int. Cl. B01D 53/62; C07D 251/54

(52) U.S. Cl. 423/228; 544/196

(57) ABSTRACT

Methods of removing carbon dioxide comprising contacting a gas mixture comprising carbon dioxide with a sorbent. The sorbent comprises the following chemical structure:

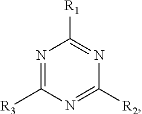

wherein at least one of R₁, R₂, and R₃ comprises a primary amine group and wherein the sorbent is not melamine and does not comprise the following chemical structure:

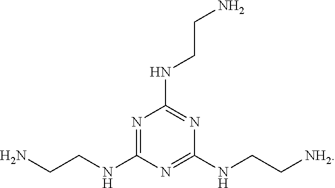

Sorbents for removing carbon dioxide are also disclosed.

### STATEMENT REGARDING FEDERALLY SPONSORED RESEARCH OR DEVELOPMENT

This invention was made with government support under Contract Number DE-AC07-051D14517 awarded by the United States Department of Energy. The government has certain rights in the invention.

### TECHNICAL FIELD

The invention, in various embodiments, relates generally to a sorbent for removing carbon dioxide (CO₂). More specifically, the invention, in various embodiments, relates to a triazine compound and methods of removing CO₂ using the triazine compound as the sorbent.

### BACKGROUND

The capture and removal of CO₂ has become a high priority for society due to growing concerns stemming from the release of CO₂ into the atmosphere. Various materials, such as amines, zeolites, or metal oxides have been proposed for use as so-called “scrubbers” to remove CO₂ from flue gas. When the CO₂ scrubber is an amine, an aqueous solution of the amine is formulated and the flue gas is contacted with the aqueous solution to remove the CO₂. Examples of amine scrubbers include ethanolamines, such as monoethanolamine (MEA), diethanolamine (DEA), and methyldiethanolamine (MDEA), alkyl or aromatic amines, piperidine derivatives, piperazine derivatives, or diazacycloheptane derivatives. In addition, amine groups attached to a surface of a solid substrate have been used to capture CO₂. The amine groups are attached by reacting an amine salt with metal salts on the surface of the solid substrate. The amine salt is a halogenated, alkyl- or aryl-substituted amine salt. The ethanolamines are able to capture CO₂ at low temperature and/or high pressure, and are regenerated, by releasing CO₂, at high temperature and/or low pressure. One disadvantage of using ethanoloamines is their corrosive nature, which causes corrosion of equipment used in the scrubbing process. Corrosion inhibitors are used in the scrubbing process to enable the use of conventional construction materials, which reduces costs associated with the scrubbing process. Another disadvantage is the loss of the liquid amine through degradation or volatilization. For industries having high concentrations of CO₂ in the flue gas, such as cement production (14-33% CO₂ in the flue gas) compared to electricity generation (4-14% CO₂ in the flue gas), the flue gas is cycled through the ethanoloamine multiple times.

A melamine-formaldehyde adsorbent has also been investigated for use to capture about 4% by weight CO₂ at 25° C. To improve the CO₂ capture, the melamine-formaldehyde film is mixed with potassium carbonate (K₂CO₃) and heated to a temperature of between 400° C. and 700° C. to activate the melamine-formaldehyde film. During activation, the melamine-formaldehyde film is pyrolyzed to an ash having a surface area of up to about 56 m²/g. The pyrolysis causes amine groups of the melamine to be eliminated as ammonia, resulting in a structure that includes a triazine ring. As a result of the ammonia elimination, the melamine-formaldehyde adsorbent has decreased basicity and decreased CO₂ uptake.

Melamine-type dendrimers have also been investigated for the adsorption of CO₂. The melamine-type dendrimers are attached to mesocellular siliceous foam by reacting the mesocellular siliceous foam with 2,3,6-trichlorotriazine and ethylenediamine.

It would be desirable to remove CO₂ using a sorbent that has a high capacity for CO₂, is regenerated easily, and does not corrode equipment. It would also be desirable for the sorbent to be a solid material, reducing the loss of the sorbent due to volatilization.

### BRIEF SUMMARY

An embodiment of the present disclosure includes a method of removing carbon dioxide comprising contacting a gas mixture comprising carbon dioxide with a sorbent. The sorbent comprises the following chemical structure:

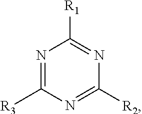

wherein at least one of R₁, R₂, and R₃ comprises a primary amine group and wherein the sorbent is not melamine and does not comprise the following chemical structure:

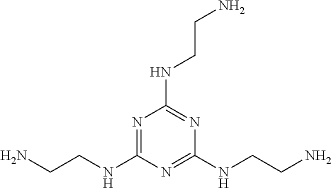

(2,4,6-tris(ethylenediamine)-1,3,5-triazine).

Another embodiment of the present disclosure includes a method of removing carbon dioxide comprising flowing a gas mixture comprising carbon dioxide through a sorbent associated with a combustion apparatus. The sorbent consists of a triazine compound having the chemical structure shown above, wherein the sorbent is not melamine.

Yet another embodiment of the present disclosure includes a sorbent comprising a triazine compound having the chemical structure shown above, wherein the sorbent is not melamine or 2,4,6-tris(ethylenediamine)-1,3,5-triazine.

Still yet another embodiment of the present disclosure includes a sorbent consisting of a triazine compound having the chemical structure shown above, wherein each of R₁, R₂, and R₃ is independently selected from the group consisting of X—NH₂, X—Y—NH₂, X—Z, or X—Y—Z, wherein X comprises oxygen, nitrogen, carbon, or sulfur, Y comprises an alkyl group having between one carbon atom and eight carbon atoms, and Z comprises a primary amine precursor group.

### BRIEF DESCRIPTION OF THE SEVERAL VIEWS OF THE DRAWINGS

While the specification concludes with claims particularly pointing out and distinctly claiming that which is regarded as the present invention, the advantages of this invention may be more readily ascertained from the following description of the invention when read in conjunction with the accompanying drawings in which:

FIG. 1 is a schematic illustration of possible binding mechanisms of CO₂ to a triazine compound according to an embodiment of the present disclosure;

FIG. 2 is a carbon dioxide sorption/desorption plot for a triazine compound (Material 1) according to an embodiment of the present disclosure;

FIG. 3 is a carbon dioxide sorption/desorption plot for a triazine compound (Material 2) according to an embodiment of the present disclosure; and

FIG. 4 is a carbon dioxide sorption/desorption plot for a triazine compound (Material 3) according to an embodiment of the present disclosure.

### DETAILED DESCRIPTION

A sorbent for removing or sequestering CO₂ from a gas mixture that includes CO₂ is disclosed, as is a method of removing CO₂ from the gas mixture utilizing the sorbent. The sorbent may be a solid at room temperature and at a temperature at which the sorbent is used, such as a triazine compound having the following general chemical structure:

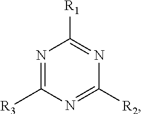

where R₁, R₂, and R₃ are the same or different moieties. R₁, R₂, and R₃ are also referred to herein as pendant groups. At least one of R₁, R₂, and R₃ includes a primary amine group (—NH₂) at a terminal portion thereof or a functional group that may be converted to a primary amine group at a terminal portion thereof, the latter of which is referred to herein as a primary amine precursor group. In one embodiment, each of R₁, R₂, and R₃ includes at least one primary amine group, at least one primary amine precursor group, or combinations thereof at the terminal portion thereof. By appropriately selecting the R₁, R₂, and R₃ groups to include a plurality of primary amine groups or primary amine precursor groups, the ability of the triazine compound to bind CO₂ may be tailored.

As used herein, the terms “comprising,” “including,” “containing,” “characterized by,” and grammatical equivalents thereof are inclusive or open-ended terms that do not exclude additional, unrecited elements or method acts, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the invention and such term is used in preference to the more restrictive term “is” so as to avoid any implication that other, compatible materials, structures, features and methods usable in combination therewith should or must be, excluded.

Each of the pendant groups may include a carbon atom or a heteroatom, such as a nitrogen atom or an oxygen atom, through which the pendant group is bound or attached to a backbone of the triazine compound. The atom attaching the pendant group to a carbon atom of the triazine compound is referred to herein as “X” and is described in more detail below. The pendant groups may also include the primary amine group or the primary amine precursor group, which is referred to herein as “Z” and is described in more detail below. The primary amine group or the primary amine precursor group may be positioned at the terminus of the pendant group, in a position furthest away from the triazine backbone. The X atom and the primary amine group or the primary amine precursor group may, optionally, be separated from one another by a linker group, which is referred to herein as “Y” and is described in more detail below. Accordingly, each of the pendant groups R₁, R₂, and R₃ may be represented by one of the following general formulas: X—NH₂, X—Y—NH₂, X—Z, or X—Y—Z, where each of the pendant groups at the R₁, R₂, and R₃ positions of the triazine compound is independently selected. General chemical structures of exemplary triazine compounds that are contemplated for use as the sorbent are shown below, where the pendant groups are positioned at each of the R₁, R₂, and R₃ positions:

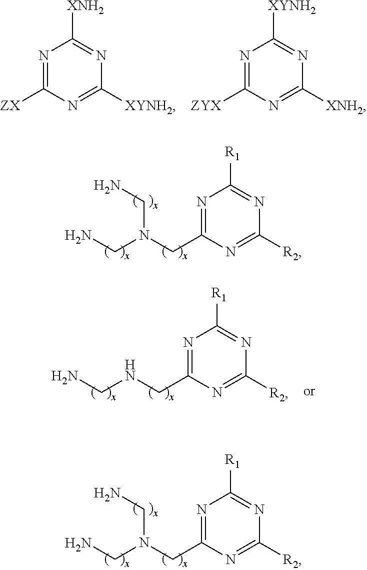

where X, Y, Z, R₁, and R₂ are as described below and x is an integer from 1 to 3. For simplicity, in some of the triazine compounds, only one pendant group is shown attached to the R₃ position of the triazine compound. The other pendant groups (R₁ and R₂) on those triazine compounds may be the same as the illustrated pendant group or different. Therefore, the triazine compound may be a symmetrical or asymmetrical compound having different combinations of pendant groups than those illustrated above.

The sorbent's affinity for CO₂ may be tailored by adjusting the number of nitrogen atoms in the triazine compound, such as the number of nitrogen atoms in the pendant groups. By way of example, by increasing the number of nitrogen atoms in the pendant groups of the triazine compound, the sorbent's affinity for CO₂ may be increased. For simplicity and convenience, the nitrogen atoms in the triazine compound are referred to herein as ring nitrogen atoms, pendant nitrogen atoms, or primary amine nitrogen atoms. As used herein, the term “ring nitrogen atom” means and includes a nitrogen atom in the heterocyclic ring of the triazine backbone. As used herein, the term “pendant nitrogen atom” means and includes a nitrogen atom in the X portion, Y portion, or Z portion of the pendant group. As used herein, the term “primary amine nitrogen atom” means and includes a nitrogen atom at the terminal portion of the pendant group.

By way of example, the triazine compound may include, but is not limited to, one of the following compounds:

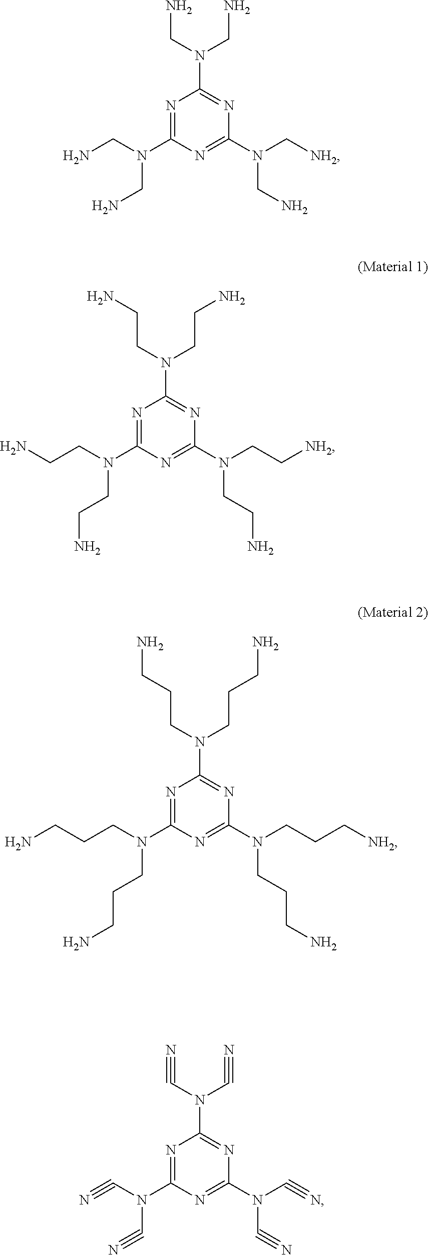

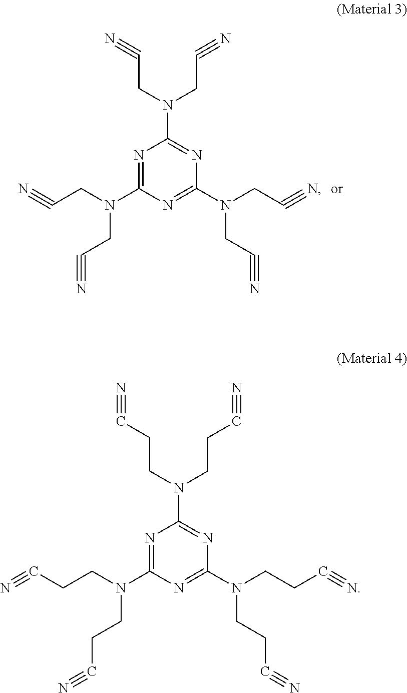

The triazine compound may be a solid, such as a powder, polymer, or polymer foam, at room temperature and at a temperature at which the triazine compound is used as the sorbent. The triazine compound may be substantially non-porous, such as having a surface area of less that approximately 1.0 m2/g. By utilizing a solid material as the sorbent, the corrosiveness of the sorbent may be reduced relative to that of liquid amines, such as ethanoloamines. In addition, corrosive inhibitors may not need to be used. Furthermore, since the triazine compound is a solid material, loss of the material due to volatilization is reduced relative to that of the liquid amines, such as ethanoloamines. Since the triazine compound is a solid, the triazine compound may be regenerated at a lower temperature than conventional liquid amines. While the triazine compound may be a liquid, depending on the pendant groups attached to the triazine backbone, regeneration of such a sorbent may be harder since the liquid may be hydroscopic. By using the triazine compound as a sorbent, CO₂ may be more effectively removed from a gas mixture than by using conventional liquid amines. Additionally, less of the triazine compound may be used to remove a particular amount of CO₂ relative to the amount of a conventional liquid amine needed.

While the sorbent is described above as having a triazine backbone, the sorbent may include another substituted, nitrogen-containing heterocyclic compound, such as a substituted, aromatic nitrogen-containing heterocyclic compound or a substituted, aliphatic nitrogen-containing heterocyclic compound. The substituted, nitrogen-containing heterocyclic compound may include a nitrogen-containing backbone with substituents attached to the carbon atoms of the nitrogen-containing backbone. The nitrogen-containing heterocyclic compound may be an azolidine compound, a pyrrole compound, an imidazolidine compound, a pyrazolidine compound, an imidazole compound, an imidazoline compound, a pyrazole compound, a pyrazoline compound, a triazole compound, a tetrazole compound, a piperidine compound, a pyridine compound, a piperazine compound, a diazine compound, such as pyrazine, pyrimidine, or pyridazine, a tetrazine compound, an azepane compound, an azepine compound, a diazepine compound, an azocane compound, or an azocine compound. The substituents on the substituted, nitrogen-containing heterocyclic compound may be the same as the pendant groups described above in regard to the triazine compound.

The triazine compound may be synthesized by reacting a triazine precursor material with at least one nucleophilic compound. The nucleophilic compound may be a precursor or reagent for the pendant group to be added to the triazine precursor material. The triazine precursor material and the nucleophilic compound may be commercially available materials, such as from Sigma-Aldrich Co. (St. Louis, Mo.), or may be synthesized by conventional techniques. The triazine precursor material and the nucleophilic compound may be commercially available in large quantities and may be relatively inexpensive. For instance, the triazine compound may be synthesized by reacting cyanuric chloride (2,4,6-trichloro-1,3,5-triazine) with the at least one nucleophilic compound. The nucleophilic compound may be a chemical compound having one of the following general chemical formulas as a portion thereof: X—NH₂, X—Y—NH₂, X—Z, or X—Y—Z, where X is the atom that attaches the pendant group to the ring carbon atom of the triazine backbone, Y (if present) is the linker group, and Z is the functional group that may be converted to a primary amine group. The X atom may be a nucleophilic atom, such as nitrogen, oxygen, or sulfur. However, X may also be a carbon atom that is more nucleophilic than the ring carbon atom of the triazine backbone.

The nucleophilic compound may be a precursor or reagent of the pendant group to be substituted on the triazine precursor material, such as cyanuric chloride. The chemical structure of the nucleophilic compound may be selected such that reaction with the cyanuric chloride produces the desired triazine compound. The linker group (Y) of the nucleophilic compound, if present, may be an alkyl group having between one carbon atom and eight carbon atoms, such as between one carbon atom and four carbon atoms. The alkyl group may be a straight or branched hydrocarbon chain. Adjusting the number of carbon atoms in the linker group to between one carbon atom and eight carbon atoms may enable the sorbent's affinity for CO₂ to be tailored. For instance, the greater the number of carbon atoms in the linker group, the greater the flexibility of the pendant groups, which may enable increased binding between the sorbent and the CO₂. However, if the number of carbon atoms in the linker group is greater than eight carbon atoms, the CO₂ binding may decrease due to an increased distance between the nitrogen atom of the primary amine group and the ring nitrogen atoms, or the nitrogen atom of the primary amine group and the atom X attaching the pendant group to the triazine backbone. In addition, if the linker group includes too many carbon atoms, the primary amine groups at the terminal portion of the pendant groups may be spaced further apart from one another, decreasing the ability of the pendant group to bind to more than one molecule of CO₂. The linker group may, optionally, include at least one heteroatom, such as nitrogen, oxygen, or sulfur. By way of example, the linker group may include a secondary amine group, a tertiary amine group, or combinations thereof. The functional group (Z) that may be converted to the primary amine group may be a nitrile group, a carboxylic acid group, a primary alcohol group, a secondary alcohol group, an alkyl halide group.

By way of example, the nucleophilic compound may be an alkylamine compound, such as ethylenediamine, diethylenetriamine, or N,N-dimethylethylenediamine. The nucleophilic compound may also be an aminoalcohol compound If the atom X attaching the pendant group to the triazine backbone is an oxygen atom. The nucleophilic compound may be reacted with the cyanuric chloride to form a triazine compound substituted with the corresponding alkylamine group or aminoalcohol group. The nucleophilic compound may also be a nitrile compound, such as iminodiacetonitrile, iminodipropionitrile, or aminoacetonitrile, each of which may be reacted with the cyanuric chloride to form a triazine compound substituted with the corresponding nitrile group. By utilizing different nucleophilic compounds, the triazine compound having the desired pendant groups may be formed. The appropriate selection of the pendant groups may enable the physical properties of the triazine compound to be tailored, as well as providing the triazine compound with the desired number of nitrogen atoms to optimize the triazine compound's ability to bind CO₂.

During the synthesis of the triazine compound, a nucleophilic addition occurs in which the chlorine atoms of the cyanuric chloride are substituted with the pendant groups. The nucleophilic compound may be added to a solution of the cyanuric chloride so that the nucleophilic compound is sequentially reacted with each of the three ring carbon atoms of the cyanuric chloride. If the three pendant groups on the triazine compound are to be the same, an excess of the nucleophilic compound may be reacted with the cyanuric chloride. By way of example, from approximately three molar equivalents to approximately eight molar equivalents of the nucleophilic compound may be used relative to the cyanuric chloride. If the triazine compound is to include three different pendant groups, approximately one molar equivalent of a first nucleophilic compound may be reacted with the cyanuric chloride to attach a first pendant group thereto, approximately one molar equivalent of a second nucleophilic compound may be reacted with the cyanuric chloride to attach a second pendant group thereto, and from approximately one molar equivalent to approximately three molar equivalents of a third nucleophilic compound may be reacted with the cyanuric chloride to attach a third pendant group thereto. Since the three chlorine atoms of the cyanuric chloride have different reactivities, different nucleophilic compounds may be reacted with the cyanuric chloride to produce an asymmetric triazine compound, i.e. a triazine compound having at least two different pendant groups attached to the triazine backbone.

The cyanuric chloride may be dissolved or suspended in a solvent, such as ethanol, tetrahydrofuran (THF), 1,4-dioxane, or other appropriate organic solvent. The solvent may be selected based on the pendant groups to be attached to the triazine backbone and the solubility of the cyanuric chloride or of intermediate reaction products in the solvent. The reaction may be conducted in the presence of a base, such as N-ethyldiisopropylamine (DIPEA). The amount of base used may depend on the number of chlorine atoms on the cyanuric chloride. For each chlorine atom, a minimum of one molar equivalent of base may be used. Since cyanuric chloride includes three chlorine atoms, a minimum of three molar equivalents of the base may be used. By adjusting the temperature conditions under which the reaction is conducted, the nucleophilic compound may be reacted with each of the three ring carbon atoms, enabling different pendant groups to be attached to the triazine backbone. The reaction may be conducted at a temperature ranging from approximately 0° C. to approximately 100° C. depending on the solvent used and the pendant groups to be attached to the triazine backbone. Since the reactivity of the ring carbon atoms decreases as each chlorine atom is sequentially replaced with the pendant group, the reaction temperature may be increased in a stepwise manner as the reaction proceeds to ensure that the pendant groups are attached to all three ring carbon atoms in the triazine backbone. By way of example, the reaction temperature may be raised in a stepwise manner from 0° C. to 25° C. to 67° C. to form the triazine compound having three pendant groups.

As each pendant group is attached to the triazine backbone, the triazine backbone becomes more deactivated such that the third pendant group is harder to attach than the first two pendant groups. The reaction time may also be increased if the third pendant group is difficult to attach. The nucleophilic compound and the triazine precursor material, such as cyanuric chloride, may be reacted for an amount of time sufficient for the desired pendant groups to attach to the triazine backbone, such as from approximately one hour to approximately twenty-four hours. The resulting triazine compound may be removed from excess starting materials, such as by washing the reaction mixture with an acid wash or a base wash to remove excess base and triazine precursor material. The triazine compound may be produced at a yield of between about 50% and about 90%.

If the pendant group includes the primary amine precursor group, such as a nitrile group, the primary amine precursor group may be converted to a primary amine group before using the triazine compound as the sorbent. By way of example, if the primary amine precursor group is a nitrile group, the nitrile group may be hydrogenated to produce the primary amine group. Hydrogenation of the nitrile group may be conducted by conventional techniques, which are not described in detail herein.

The synthesis of the triazine compound may also utilize protecting groups to prevent multiple primary amine groups or multiple secondary amine groups in the triazine compound from crosslinking and forming extended structures. If the nucleophilic compound includes a primary amine group, the nucleophilic compound may be reacted with a protecting group to protect the primary amine group. Protecting groups for primary amines are known in the art and, therefore, are not described in detail herein. By way of example, the protecting group may include, but is not limited to, 4-methyl-2-pentanone, benzaldehyde, or ethyl trifluoroacetate. The protecting group may form a Schiff-base with the primary amine group of the nucleophilic compound, protecting the primary amine group without affecting other amine groups in the nucleophilic compound, such as secondary amine groups. The nucleophilic compound having the protected primary amine group may then be reacted with the cyanuric chloride, forming a triazine compound in which the primary amine groups are protected by the protecting groups. Following the nucleophilic addition, the protecting group may be removed, such as by hydrolysis, to produce the triazine compound. By protecting the primary amine groups, the pendant groups attached to the triazine backbone may include any number of primary amine groups, increasing the loading of primary amine groups in the triazine compound and providing a large number of binding sites for CO₂ capture.

The triazine compound of the present disclosure is a solid and has a low surface area (approximately 0.88 m2/g), which may limit the ability of CO₂ to bind to the triazine compound. To increase the CO₂ binding, the triazine compound may be attached to a solid support having a high surface area, such as a surface area of greater than or equal to approximately 100 m²/g. By way of example, the solid support may include, but is not limited to, silica, a mesosilicate, or alumina. The triazine compound may be attached to the solid support through one of the pendant groups. Of the three pendant groups of the triazine compound, one may be attached to the solid support, while the other two may include primary amine groups that are configured to bind CO₂. The higher surface area of the solid support having the triazine compound attached thereto may expose additional functional groups to the CO₂, increasing the amount of CO₂ binding. By increasing the accessibility of the triazine compound to the CO₂, such as by increasing the surface area, the triazine compound may be configured to bind to additional CO₂.

The sorbent (i.e., the triazine compound) may be used to remove CO₂ from a gas mixture that includes CO₂ by contacting the sorbent with the gas mixture. The gas mixture may be flowed through the sorbent, enabling the CO₂ to complex with the triazine compound. To enable the CO₂ and the triazine compound to react, the gas mixture and triazine compound may be contacted at a temperature of from approximately room temperature (20° C.-25° C.) to less than or equal to approximately 60° C., at which temperature the triazine compound is a solid.

In addition to CO₂, the gas mixture may include hydrogen sulfide (H₂S), nitrogen (N₂), oxygen (O₂), water (H₂O), carbon monoxide (CO), carbon disulfide (CS₂), hydrogen cyanide (HCN), carbonyl sulfide (COS), a sulfur oxide (SO_{X}), such as SO₂ or SO₃, or a nitrogen oxide (NOₓ), such as NO or NO₂. The gas mixture may be produced as a result of iron production, steel production, cement production, chemical production, oil refining, electricity generation, or other industrial process in which CO₂ is produced. The gas mixture may be a flue gas or other CO₂-containing gas. As used herein, the term “flue gas” means and includes a gas that exits to the atmosphere through a flue. The flue gas may be an exhaust gas from a combustion apparatus, such as an exhaust gas from a fireplace, oven, furnace, boiler, steam generator, power plant, or other apparatus in which a combustion process is conducted. The flue gas may be produced by a combustion process and may include nitrogen, CO₂, water vapor, oxygen gas (O2), CO, a NOₓ, a SOₓ, and particulates, such as soot or hydrocarbon compounds. The sorbent may be located in, on or proximate to the combustion apparatus near an orifice through which the gas mixture exits the combustion apparatus. The combustion apparatus may include a container near the exit thereof to contain the sorbent. Alternatively, the sorbent may be contained in an apparatus separate from the combustion apparatus. The gas mixture produced by the combustion apparatus may be transported from the combustion apparatus and into the apparatus containing the sorbent.

Upon contact, the CO₂ may react with the sorbent, producing a complex of the CO₂ and the sorbent. Without being bound by any theory, it is believed that any of the nitrogen atoms in the triazine compound may interact with the CO₂. While the primary amines in the triazine compound may provide a majority of the CO₂ uptake, the ring nitrogen atoms and the pendant nitrogen atoms may interact, albeit to a lesser extent, with the CO₂. Due to the large number of nitrogen atoms present in the triazine compound, the triazine compound may have numerous modes of binding CO₂. By way of example, the material described below in Example 1 as Material 1 includes six primary amine groups and six secondary or tertiary amine groups, for a total of twelve nitrogen atoms. If, however, the pendant groups are bound to the triazine backbone by an oxygen atom or a carbon atom and each pendant group includes a single primary amine group, the triazine compound may include a minimum of three primary amine groups and three tertiary amine groups, for a total of six nitrogen atoms.

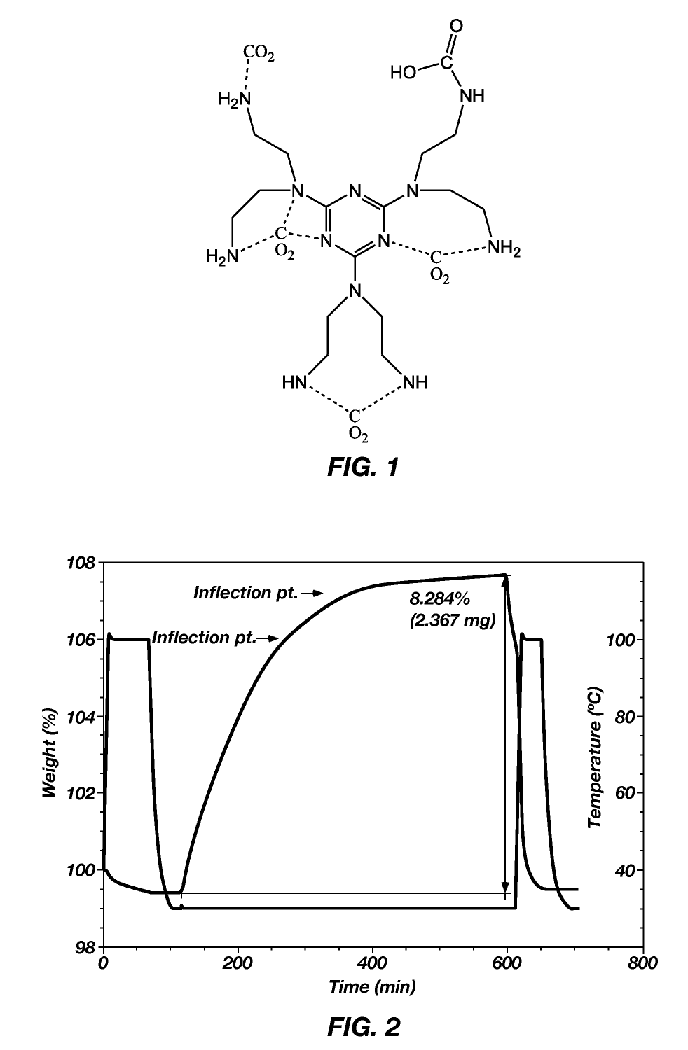

Possible binding mechanisms of CO₂ to an exemplary triazine compound are shown in FIG. 1, where the triazine compound is the material described in Example 1 as Material 1. However, FIG. 1 does not show all of the binding mechanisms between CO₂ and Material 1. The possible binding mechanisms include covalent bonding (i.e., carbamate formation), electrostatic interactions, or van der Waals interactions. As shown in FIG. 1, each molecule of the triazine compound may bind to multiple CO₂ atoms due to the large number of nitrogen atoms present. The sorption of the CO₂ from the gas mixture may be due to chemisorption, which provides selectivity for CO₂ relative to other gases in the gas mixture, such as N₂. If melamine were to be used as the sorbent, the sorbent may exhibit a flow of electron density from the pendant nitrogen atom to the triazine backbone, forming a partial double bond. Thus, the sorbent may exhibit electron delocalization between the ring nitrogen atoms and the pendant nitrogen atoms. However, by attaching the pendant groups to the triazine backbone, where the pendant groups are as previously described and include the primary amine groups at the terminus thereof, the triazine compound of the present disclosure may be tailored to bond to CO₂.

As CO₂ complexes to the multiple binding sites of the triazine compound, the sorbent may become deactivated once a majority of binding sites on the triazine compound are bound to the CO₂. Once the triazine compound is loaded (i.e., deactivated) with CO₂, the triazine compound may be regenerated by exposing the deactivated triazine compound to heat. Upon heating the triazine compound, the CO₂ may be released from the complex, enabling the triazine compound to be regenerated. Since the sorbent is selective for CO₂, the CO₂ regenerated from the triazine compound may be substantially pure CO₂. The CO₂ released from the sorbent may be collected and sequestered by conventional techniques. Alternatively, the CO₂ released from the sorbent may be used as a CO₂ source in an industrial process. To regenerate the sorbent, the deactivated triazine compound may be exposed to a temperature of less than or equal to approximately 100° C. for an amount of time sufficient to desorb the CO₂ therefrom. The deactivated triazine compound may be regenerated by exposing the sorbent to a temperature of from approximately 55° C. to less than or equal to approximately 100° C. The exposure time may range from approximately five minutes to approximately one hundred eighty minutes depending on the temperature to which the deactivated triazine compound is exposed and the extent of regeneration desired. By way of example, heating the deactivated sorbent to a temperature of approximately 80° C. for approximately ninety minutes may remove substantially all of the CO₂, such as approximately 98% of the CO₂. Since the sorbent may be regenerated at a relatively low temperature, the costs associated with the regeneration process are low. Thus, the regeneration of the sorbent (i.e., removal of the CO₂) may be cost-effective since the bulk of the energy expenditure for a sorbent is during the regeneration process. The triazine compound may be used to remove CO₂ and then regenerated multiple times without degradation. The regenerated triazine compound may then be used again as a sorbent to remove CO₂.

### EXAMPLES

### Example 1

### Synthesis of Materials 1 and 3

Cyanuric chloride and iminodiacetonitrile were reacted to produce the corresponding nitrile designated above as Material 3, as shown below:

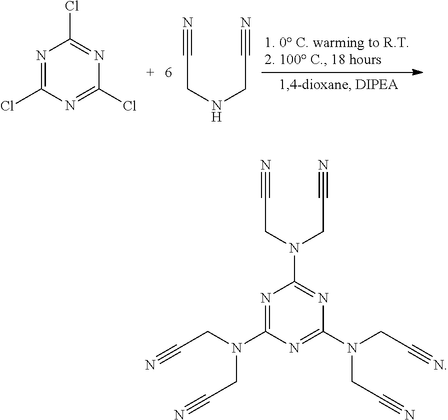

Two equivalents of iminodiacetonitrile were added to a solution of cyanuric chloride in 1,4-dioxane at 0° C. The reaction mixture was allowed to warm to room temperature (between approximately 20° C. and approximately 25° C.) and stirred overnight. The following day, four additional equivalents of iminodiacetonitrile were added and the reaction refluxed at 100° C. for 18 hours. The precipitate in the reaction mixture was filtered and washed with 1,4-dioxane to remove unreacted iminodiacetonitrile. The product was then extracted with acetone, producing Material 3, which was confirmed by ¹³C NMR.

Material 3 was then hydrogenated into Material 1 (2,4,6-tris(iminodiethylamine)-1,3,5-triazine). To hydrogenate the nitrile groups of Material 3 into primary amine groups, a Shaker Type 3911 Hydrogenation Apparatus (Parr Instrument Co.) was used. A conventional Raney nickel was used as the catalyst. The hydrogenation reaction was conducted in the presence of the Raney nickel at 30 psi of hydrogen, 1M NaOH, and a water/1,4-dioxane mixture or water/THF mixture, yielding approximately 84% of Material 1. The water/1,4-dioxane mixture or water/THF mixture included five parts water to three parts 1,4-dioxane or THF. Material 1 was a tan powder and had a measured surface area of 0.88 m²/g.

### Example 2

### Synthesis of Materials 2 and 4

Cyanuric chloride and iminodipropionitrile were reacted to produce the corresponding nitrile designated above as Material 4, as shown below:

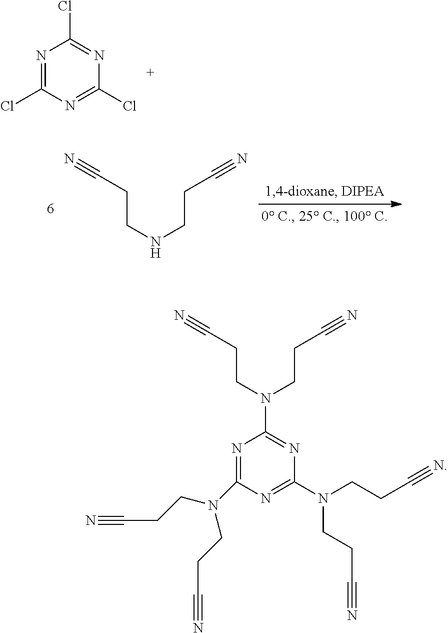

Two equivalents of iminodipropionitrile were added to a solution of cyanuric chloride in 1,4-dioxane at 0° C. The reaction mixture was allowed to warm to room temperature (between approximately 20° C. and approximately 25° C.) and stirred overnight. The following day, four additional equivalents of iminodiacetonitrile were added and the reaction refluxed at 100° C. for 18 hours. The precipitate in the reaction mixture was filtered and washed with 1,4-dioxane to remove unreacted iminodiacetonitrile. The product was then extracted with acetone, producing Material 4, which was confirmed by ¹³C NMR.

Material 4 was then hydrogenated into Material 2 (2,4,6-tris(iminodipropylamine)-1,3,5-triazine). To hydrogenate the nitrile groups of Material 3 into primary amine groups, a Shaker Type 3911 Hydrogenation Apparatus (Parr Instrument Co.) was used. A conventional Raney nickel was used as the catalyst. The hydrogenation reaction was conducted in the presence of the Raney nickel at 30 psi of hydrogen, 1M NaOH, and a water/1,4-dioxane mixture, yielding Material 2. The water/1,4-dioxane mixture included five parts water to three parts 1,4-dioxane. Material 2 was a tan powder.

### Example 3

Testing of CO₂ Uptake and Regeneration

Carbon dioxide sorption/desorption testing on Materials 1-3 was performed using a TA Instruments Q500 thermogravimetric analysis (TGA). Samples of each of Materials 1-3 were heated in the presence of flowing nitrogen to remove any moisture or CO₂ present in the samples. The samples were cooled to 30° C. and the nitrogen flow was switched to a flow of CO₂. The weight of the samples was measured initially and then the weight change in the samples (due to absorption of CO₂) was measured after a set period of time. Table 1 shows the percent weight gain at different CO₂ flow rates for Materials 1-3.

TABLE 1
Percent weight gain due to CO₂ uptake.

| | CO₂ Flow Rate | |
|---|---|---|
| Material | (ml/min) | % weight gain |
| 1 | 60 | 0.3728^{a} |
| 1 | 80 | 1.030^{a} |
| 1 | 100 | 1.850^{a} |
| 1 | 120 | 2.276^{a} |
| 1 | 140 | 2.732^{a} |
| 1 | 160 | 2.975^{a} |
| 1 | 160 | 8.284^{b} |
| 1 | 180 | 2.878^{a} |
| 1 | 200 | 3.006^{a} |
| 2 | 160 | 5.647^{b} |
| 3 | 160 | 0.0075^{b} |

^{a}material hydrogenated using water/THF
^{b}material hydrogenated using water/1,4-dioxane

As shown in Table 1, Material 1 had a weight gain of 8.284 wt % compared to a weight gain of 5.647 wt % for Material 2, an increase of approximately 46%, just due to having one less carbon in the linker group of the triazine compound. Material 3, the nitrile precursor to Material 1, had a weight gain of 0.0075 wt %. Material 3, which lacked primary amine groups, had a significantly lower weight gain than Material 1 or Material 2, suggesting the primary amine groups in Material 1 and Material 2 are important to CO₂ capture. While there was uptake of CO₂ with Material 3, the amount was low relative to Material 1 and Material 2. Therefore, it is believed that while the ring nitrogen atoms and the pendant nitrogen atoms may participate in CO₂ capture, they do not participate to a significant degree without primary amine groups present.

Using Material 1, the flow of CO₂ was then switched back to a flow of nitrogen, the samples were heated stepwise to a temperature of 60° C., 80° C., and 100° C. over a time period of approximately 90 minutes, and the weight change in the samples (due to release of CO₂) was measured. Table 2 shows the regeneration of Material 1 at 60° C., 80° C., and 100° C.

TABLE 2
Regeneration testing for Material 1.

| Temperature | |
|---|---|
| (° C.) | % CO₂ removed in 90 minutes |
| 60 | 85 |
| 80 | 98 |
| 100 | 100 (less than 90 minutes) |

As shown in Table 2, Material 1 was substantially regenerated (98% of CO₂ removed) after heating to 80° C. for 90 minutes. In the first twenty-four minutes after switching from CO₂ to N₂, 68% of the captured CO₂ was removed from Material 1. After twenty-four minutes, the rate of CO₂ release decreased. At temperatures of 60° C. and 80° C., the release of CO₂ ended when the temperature was reduced to 30° C. after 90 minutes. However, the curves had not leveled off yet, indicating more of the CO₂ may be removed at those temperatures with a longer heat time.

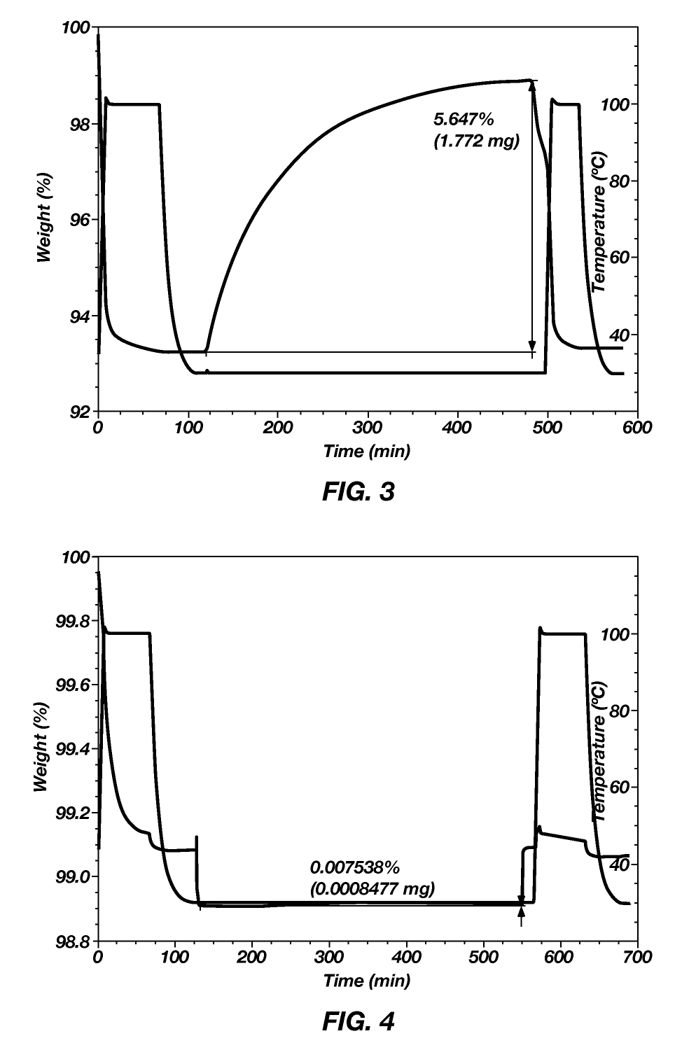

Carbon dioxide sorption/desorption plots for Materials 1-3 are shown in FIGS. 2-4, respectively. The plots included two inflection points, which are believed to be changes in binding mechanism. At the inflection points, all of the sites available for low energy electrostatic binding may have been filled, leaving behind higher energy sites that take longer to fill due to the energy requirements. The inflection points may also indicate penetration of CO₂ into the triazine compound, which should be a slower process that absorption.

Even with a low surface area, Material 1 had comparable or improved levels of CO₂ binding compared to conventional CO₂ sorbents, as shown in Table 3. The conventional CO₂ sorbents include monoethanolamine (MEA), methyldiethanolamine (MDEA), and several zeolites, which capture CO₂ by physisorption due to their porous, large surface area structure. However, zeolites have no selectivity for CO₂. The comparative data for the conventional CO₂ sorbents was as reported by the National Energy Technology Laboratory. The data for Materials 1 and 2 was calculated from the TGA plots (carbon dioxide sorption/desorption plots) in FIGS. 2 and 4.

TABLE 3
Comparative CO₂ uptake data.

| Sorbent | Mole CO₂/kg sorbent |
|---|---|
| MEA | 0.85 |
| MDEA | 0.68 |
| Zeolite Z10-08 | 2.5-3 |
| Na Alumino silicate 1^{a} | 0.5-1.5 |
| Ca K Alumino silicate^{a} | 0.1-0.2 |
| Material 1 | 1.84 |
| Material 2 | 1.28 |

^{a}Physical absorption, no selectivity between gases (N₂, O₂, CO₂, SO₂, CO)

As shown above, Material 1 and Material 2 had improved CO₂ binding compared to MEA, MDEA, and Ca K alumino silicate, even though Material 1 and Material 2 have a lower surface area. Material 1 and Material 2 had comparable CO₂ binding relative to Na alumino silicate, even though Material 1 and Material 2 have a lower surface area. The difference in CO₂ binding between Material 1 and Material 2 was 0.56 mole CO₂/kg sorbent, illustrating that a change in chemical structure of the triazine compound as small as one carbon atom may impact the ability of the triazine compound to bind CO₂.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the scope of the invention as defined by the following appended claims and their legal equivalents.

What is claimed is:

1. A method of removing carbon dioxide, comprising:
contacting a gas mixture comprising carbon dioxide with a sorbent comprising the following chemical structure: 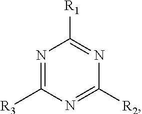
wherein at least one of R₁, R₂, and R₃ comprises a primary amine group and wherein the sorbent is not melamine and does not comprise the following chemical structure:

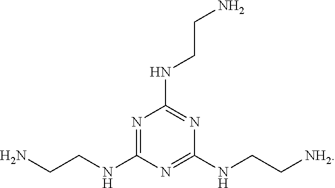

2. The method of claim 1, wherein contacting a gas mixture comprising carbon dioxide with a sorbent comprises forming a complex between the carbon dioxide and the sorbent.

3. The method of claim 1, wherein contacting a gas mixture comprising carbon dioxide with a sorbent comprises contacting a gas mixture comprising carbon dioxide and at least one of hydrogen sulfide, nitrogen, oxygen, water, carbon monoxide, carbon disulfide, hydrogen cyanide, carbonyl sulfide, a sulfur oxide, and a nitrogen oxide with the sorbent.

4. The method of claim 1, wherein contacting a gas mixture comprising carbon dioxide with a sorbent comprises contacting the gas mixture and the sorbent at a temperature of less than or equal to approximately 60° C.

5. The method of claim 1, wherein contacting a gas mixture comprising carbon dioxide with a sorbent comprises flowing the gas mixture through a solid material comprising the sorbent.

6. The method of claim 1, wherein contacting a gas mixture comprising carbon dioxide with a sorbent comprises flowing a flue gas through the sorbent.

7. The method of claim 6, wherein flowing a flue gas through the sorbent comprises flowing a flue gas comprising carbon dioxide and at least one of nitrogen, water, oxygen, carbon monoxide, a nitrogen oxide, a sulfur oxide, soot, and at least one hydrocarbon compound through the sorbent.

8. The method of claim 1, further comprising heating the triazine compound to regenerate the triazine compound.

9. The method of claim 1, wherein heating the triazine compound comprises heating the triazine compound to a temperature of less than approximately 100° C. to remove the carbon dioxide.

10. A method of removing carbon dioxide, comprising:
flowing a gas mixture comprising carbon dioxide through a sorbent associated with a combustion apparatus, the sorbent consisting of a triazine compound having the following chemical structure: 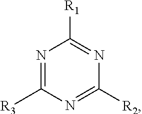
wherein at least one of R₁, R₂, and R₃ comprises a primary amine group and wherein the triazine compound is not melamine; and
reacting the carbon dioxide with the sorbent.

11. A sorbent, comprising:
a triazine compound having the following chemical structure: 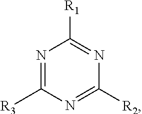
wherein at least one of R₁, R₂, and R₃ comprises a primary amine group and wherein the triazine compound is not melamine and does not comprise the following chemical structure:

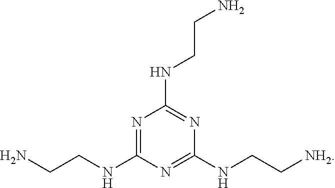

12. The sorbent of claim 11, wherein the triazine compound is a solid at room temperature.

13. The sorbent of claim 11, wherein the triazine compound is a compound having the following chemical structure: 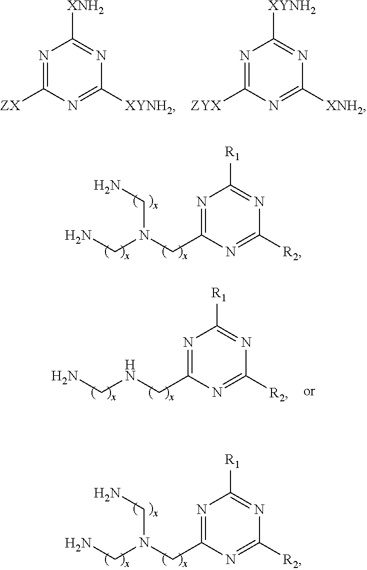
wherein X comprises oxygen, nitrogen, carbon, or sulfur, Y comprises an alkyl group having between one carbon atom and eight carbon atoms, Z comprises a primary amine precursor group, x is an integer from 1 to 3, and at least one of R₁ and R₂ comprises a primary amine group.

14. The sorbent of claim 11, wherein the triazine compound is a compound comprising the following chemical structure:

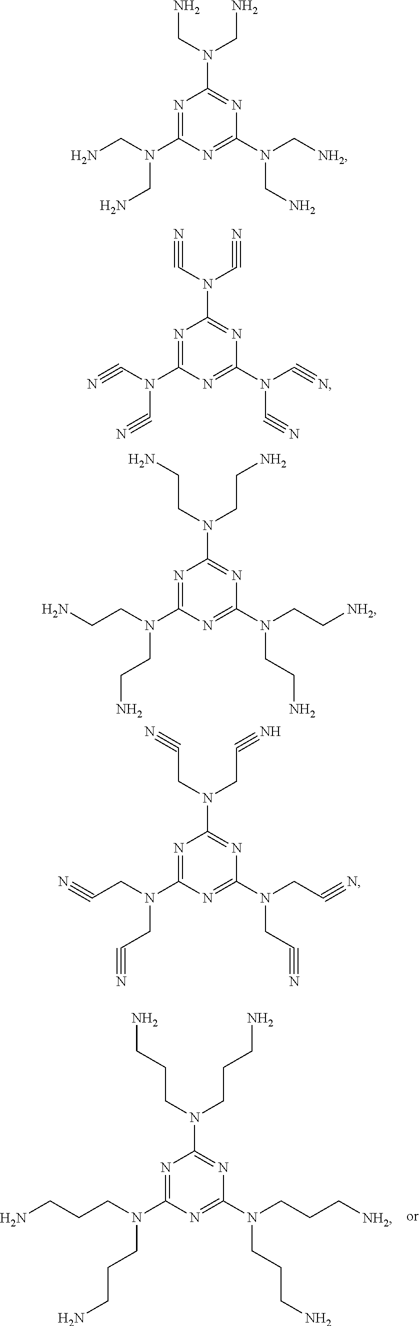

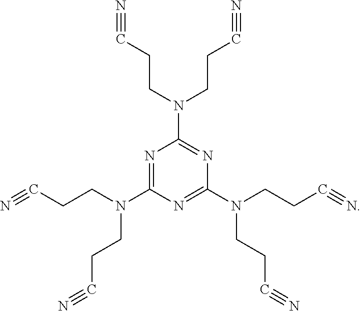

15. The sorbent of claim 11, further comprising a solid support to which the triazine compound is attached.

16. The sorbent of claim 15, wherein each molecule of triazine compound is attached to a single solid support.

17. A sorbent, consisting of:
a triazine compound having the following chemical structure: 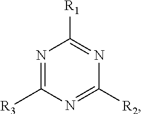
wherein each of R₁, R₂, and R₃ is independently selected from the group consisting of X—NH₂, X—Y—NH₂, X—Z, or X—Y—Z, wherein X comprises oxygen, nitrogen, carbon, or sulfur, Y comprises an alkyl group having between one carbon atom and eight carbon atoms, and Z comprises a primary amine precursor group.

18. The sorbent of claim 17, wherein the sorbent comprises a powder.

19. The sorbent of claim 17, wherein Z comprises a nitrile group, a carboxylic acid group, a primary alcohol group, a secondary alcohol group, or an alkyl halide group.

20. The sorbent of claim 17, wherein the triazine compound comprises at least six nitrogen atoms.

21. The sorbent of claim 17, wherein the triazine compound comprises between six nitrogen atoms and twelve nitrogen atoms.

22. The sorbent of claim 17, wherein the triazine compound comprises 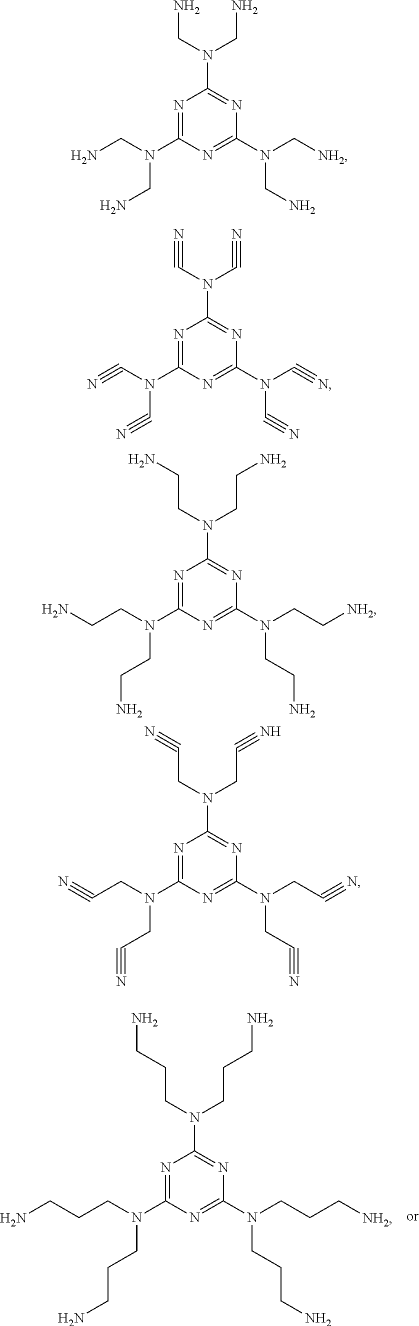 [chemical structure]
